(19) United States
(12) Patent Application Publication
(10) Pub. No.: US 2022/0133467 A1
(43) Pub. Date: May 5, 2022

(54) PROSTHESIS

(71) Applicant: Harrison M. Lazarus, Salt Lake City, UT (US)

(72) Inventor: Harrison M. Lazarus, Salt Lake City, UT (US)

(21) Appl. No.: 17/516,337

(22) Filed: Nov. 1, 2021

Related U.S. Application Data
(60) Provisional application No. 63/108,613, filed on Nov. 2, 2020.

(51) Int. Cl. A61F 2/12

(52) U.S. Cl. CPC A61F 2/12; A61F 2310/00023; A61F 2210/0014

(57) ABSTRACT

Described is a prosthesis for implantation beneath the skin of a subject, the prosthesis comprising a support structure of super elastic material (e.g., nitinol), wherein the support structure is sized and shaped for augmenting, replacing, or reconstructing tissue of the subject, such as the breast. In certain embodiments, the prosthesis further includes an elastomeric outer shell having a cavity therein, the outer shell being sized and shaped for augmenting or replacing, for example, breast tissue of the subject; wherein the support structure is disposed within the cavity of the elastomeric shell.

### CROSS-REFERENCE TO RELATED APPLICATION

This application claims the benefit under 35 U.S.C. § 119(e) of U.S. Provisional Patent Application Ser. No. 63/108,613, filed Nov. 2, 2020, and entitled “PROSTHESIS,” the disclosure of which is hereby incorporated herein in its entirety by this reference.

### TECHNICAL FIELD

The application relates to the field of medical devices and implantable prostheses generally and, in particular, to prostheses comprising super elastic material that are useful, e.g., as breast implants.

### BACKGROUND

Prostheses, such as breast prostheses, are well-known in the art and, in their most basic form, generally include a flexible shell that encloses a filler including a viscous fluid. U.S. Pat. No. 3,293,663 (Cronin), the contents of which are incorporated herein by this reference, describes a device that employs this basic structure. An overriding goal in designing and constructing such a prosthesis is to mimic, as closely as possible, the physical properties of normal breast tissue, which include, but are not limited to, density, deformability, elasticity, weight, and rigidity. A prosthesis is also preferably medically biocompatible.

Prior to 1992, silicone gel was the filler of choice for most breast prostheses, as the viscous properties of silicone gel produce a prosthesis that closely mimics the properties of normal breast tissue. Because of safety concerns, however, in 1992 the Federal Food and Drug Administration placed a moratorium on the use of silicone gels in breast implants. The moratorium led to research into alternative designs for breast prostheses, with the ultimate goal being a physiologically safer prosthesis having an appearance, consistency and feel of normal breast tissue.

Current substitutes for silicone gel include the use of: biocompatible gel compositions, such as described in U.S. Pat. No. 5,407,445 (Tautvydas et al.) and U.S. Pat. No. 5,411,554 (Scopelianos et al.); polyphasic filler materials consisting of gas-filled chambers or beads bathed in a biocompatible fluid, as described in U.S. Pat. No. 5,534,023 (Henley); partially filled hollow spheroids of polymeric material, as described in U.S. Pat. No. 6,099,565 (Sakura, Jr.); and biocompatible fluids combined with foam inserts, as described in U.S. Pat. No. 5,658,330 (Carlisle et al.) and U.S. Pat. No. 5,824,081 (Knapp, et al.) The contents of each of these patents are incorporated herein by this reference.

In contrast to the foregoing, the instant disclosure is a prosthesis that includes a super elastic material (“SEM”) (e.g., a super elastic alloy (“SEA”), or a super elastic polymer (“SEP”)). Super elastic alloys are well known in the metallurgical arts. They are used in a variety of mechanical devices, which include, for example, pipe couplings, as described in U.S. Pat. Nos. 4,035,007 and 4,198,081 (Harrison et al.); electrical connectors, as described in U.S. Pat. No. 3,740,839 (Otte et al.); and switches, as described in U.S. Pat. No. 4,205,293 (Melton et al.) super elastic alloys also have various uses in the medical field. For example, super elastic alloys have been proposed for use with intrauterine contraceptive devices, as described in U.S. Pat. No. 3,620,212 (Fannon et al.); bone plates, as described in U.S. Pat. No. 3,786,806 (Johnson et al.); and catheters, as described in U.S. Pat. No. 3,890,977 (Wilson et al.). The contents of each of these patents are incorporated herein by this reference.

Super elastic polymers are also known in the material sciences. They are used in a variety of mechanical devices, which include, for example, a fender bracket U.S. Pat. No. 9,399,490 (Aitharaju et al.); and electronics applications, such as a variable capacitor device U.S. Pat. No. 10,181,381 (Al-Hazmi et al.) super elastic polymers also have various uses in the medical field. For example, super elastic polymers have been proposed for use as in stent devices, as described in U.S. Pat. No. 5,964,771 (Beyar et al.) and orthopedic devices, as described in U.S. Pat. No. 8,277,404 (Einarsson). The contents of each of these patents are incorporated herein by this reference.

### BRIEF SUMMARY

Described herein is the use of super elastic materials in the design and construction of breast, mons pubis, buttocks, and other prostheses. In some embodiments, a super elastic material may be used as the sole structural element of the prosthesis. For example, the prosthesis may include a super elastic material support structure. In additional embodiments, the prosthesis includes an outer shell (e.g., flexible shell) and a super elastic material support structure within the outer shell. In further embodiments, the super elastic material is used conjunction with various available biocompatible filler materials, such as those described in the above-identified U.S. patents.

A super elastic material (SEM), such as a super elastic alloy (e.g., nitinol), or a super elastic polymer (e.g., polyether, polyacrylate, polyamide, polysiloxane, polyurethane, polyethylene, methyl-methacrylate (MMA), polyethylene glycol (PEG), polyethylene glycol dimethacrylate (PEGDMA), polyether amide, polyether ester, or urethane-butadiene copolymer) may take the form of a support structure (e.g., a nest structure, a web-like mesh structure, and/or a cage structure). In certain embodiments, the support structure exhibits the shape of a nest (e.g., a bird's nest). In additional embodiments, the support structure exhibits the shape of a web-like mesh. For example, the web-like mesh may resemble a typical pad of steel wool. In additional embodiments, the support structure of the super elastic material exhibits the shape of a cage structure (e.g., hollow cage structure). For example, the cage structure may resemble chicken wire manipulated to form a shape suited for a prosthesis. In additional embodiments, the support structure exhibits the shape of the nest, web-like mesh, and/or the cage structure in combination.

In some embodiments, the prosthesis includes one or more strands of a super elastic material configured to form a support structure (e.g., a nest structure, a web-like mesh structure, and/or a cage structure). In additional embodiments, the prosthesis includes a super elastic material support structure disposed within the interior volume of a flexible shell that is shaped and sized for implantation within a subject (e.g., a mammal, such as a human). In some embodiments the super elastic material support structure includes a cage structure within the interior of the flexible shell and configured to structurally support the flexible shell.

The prosthesis may then be surgically implanted within the subject (e.g., human). For example, the prosthesis may be implanted underneath a subject's skin or behind a subject's breast, buttocks, mons pubis, fascia, or pectoral muscle.

In certain embodiments, the super elastic material support structure comprises a nest or web-like mesh that substantially fills the interior volume of the flexible shell and/or the cage structure, such that any distortion of the flexible shell and/or cage structure distorts the super elastic material nest or web-like mesh in a corresponding fashion. A biocompatible gel composition, such as those disclosed in U.S. Pat. No. 5,407,445 (Tautvydas et al.) and U.S. Pat. No. 5,411,554 (Scopelianos et al.), each of which is incorporated herein by this reference, or a simple saline solution, may be added to the prosthesis following surgical implantation.

The foregoing design provides, among other things, two benefits. First, the “shape memory effect” property of the super elastic material permits flattening of a prosthesis having a normal breast-like shape prior to surgical implantation under the skin or behind the breast, fascia or pectoral muscle. Once implanted, the body heat generated by the subject will cause the super elastic material to recover its pre-flattened shape and, thereby, cause the prosthesis to recover its normal breast-like shape. This is beneficial in that a relatively small incision will be required to perform the implantation process when compared to that required for implanting a fully shaped prosthesis. A smaller incision produces less scarring and promotes faster healing following the surgical procedure.

Second, the “super elasticity” property of the super elastic material assures full recovery of the prosthesis to its normal shape following extreme manipulation of the prosthesis while behind the breast, mons pubis, buttocks, fascia, or pectoral muscle. Extreme manipulation of a prosthesis (e.g., breast prosthesis) that is constructed using a material without shape memory properties will tend to deform the material beyond the elastic limit and into the plastic range. Accordingly, the prosthesis would not return to its pre-flattened or normal shape due to the material having been plastically deformed. The super elasticity property of super elastic materials, however, provides for full recovery following deformation of the super elastic materials. For example, stresses that result in high strain of super elastic materials generally result in reversible deformation. More specifically, deformation of prostheses in accordance with the instant disclosure at body temperatures slightly above the austenitic transition temperature (for super elastic alloys), will generally be followed by automatic recovery of the pre-flattened (or normal) shape. Similarly, deformation of the prosthesis of the instant disclosure, at body temperature slightly above the recovery temperature, which is at or around the glass transition temperature, will generally be followed by automatic recovery of the pre-flattened (or normal) shape for super elastic polymers. This is beneficial in that the full shape recovery of the prosthesis may be obtained following extreme manipulation (which may occur, for example, through trauma) while under the skin and/or behind the breast, fascia, or pectoral muscle.

In certain embodiments of prostheses described herein (e.g., those without a flexible shell), the subject will be at reduced risk of developing a large cell lymphoma. Furthermore, prostheses described herein that only include a support structure (e.g., a nest structure, a mesh-like structure, and/or cage structure) will not “leak” as may be seen with prior art devices, since the support structure is open for any bodily fluid to enter or leave the interior of the support structure.

In sum, the “shape memory effect” and “super elasticity” properties of super elastic materials, in combination with the elastic properties of the flexible shell and the viscous properties of any added biocompatible gel composition or saline solution, work to provide a physiologically safe prosthesis having an appearance, consistency and feel of typical breast tissue.

### BRIEF DESCRIPTION OF THE DRAWINGS

FIG. 1 illustrates a cross-sectional view of a prosthesis following its implantation behind the breast tissue of a subject, in accordance with embodiments of the disclosure.

FIG. 2A illustrates a cross-sectional side view of a flexible shell, in accordance with embodiments of the disclosure.

FIG. 2B illustrates a cross-sectional side view of an additional flexible shell, in accordance with embodiments of the disclosure.

FIG. 3A illustrates a perspective view of a support structure, in accordance with embodiments of the disclosure.

FIG. 3B illustrates a perspective view of another support structure, in accordance with embodiments of the disclosure.

FIG. 3C illustrates a side view of an additional support structure, in accordance with embodiments of the disclosure.

FIG. 4A illustrates a cross-sectional side view of a prosthesis according to embodiments of the disclosure before being deformed to facilitate implantation.

FIG. 4B illustrates a cross-sectional side view of a prosthesis according to embodiments of the disclosure after being deformed to facilitate implantation.

FIG. 4C illustrates a cross-sectional view of a prosthesis according to embodiments of the disclosure after being implanted behind the breast tissue of a subject and following the recovery of its originally un-deformed shape.

FIG. 5A illustrates a cross-sectional side view of a prosthesis according to embodiments of the disclosure after being implanted behind the breast tissue of a subject in an un-deformed shape.

FIG. 5B illustrates a cross-sectional view of a prosthesis according to embodiments of the disclosure after being manipulated upward while implanted behind the breast tissue of a subject.

FIG. 5C illustrates a cross-sectional view of a prosthesis according to embodiments of the disclosure after returning to the originally un-deformed shape while implanted behind the breast tissue of a subject.

### DETAILED DESCRIPTION

Although the description herein predominantly relates to prosthetic breast implants, the device may alternatively be used for other prosthetic applications in the subject's (e.g., mammal's) body. For example, the prosthetic implants described herein may also refer to mons pubis implants, buttocks implants, and/or implants to facilitate tissue reconstruction (e.g., breast reconstruction following a mastectomy).

The following description provides specific details, such as material types, material thicknesses, and processing conditions in order to provide a thorough description of embodiments of the disclosure. However, a person of ordinary skill in the art will understand that the embodiments of the disclosure may be practiced without employing these specific details. Indeed, the embodiments of the disclosure may be practiced in conjunction with conventional techniques employed in the industry. Only those structures and acts necessary to understand the embodiments of the disclosure are described in detail below. Embodiments of a prosthesis as described herein may be formed by any known or conventional fabrication techniques.

Drawings presented herein are for illustrative purposes only, and are not meant to be actual views of any particular material, component, structure, device, or system. Variations from the shapes depicted in the drawings as a result, for example, of manufacturing techniques and/or tolerances, are to be expected. Thus, embodiments described herein are not to be construed as being limited to the particular shapes or regions as illustrated, but include deviations in shapes that result, for example, from manufacturing. For example, a region illustrated or described as box-shaped may have rough and/or nonlinear features, and a region illustrated or described as round may include some rough and/or linear features. Moreover, sharp angles that are illustrated may be rounded, and vice versa. Thus, the regions illustrated in the figures are schematic in nature, and their shapes are not intended to illustrate the precise shape of a region and do not limit the scope of the present claims. The drawings are not necessarily to scale. Additionally, elements common between figures may retain the same numerical designation.

As used herein, the term “configured” refers to a size, shape, material composition, material distribution, orientation, and arrangement of one or more of at least one structure and at least one apparatus facilitating operation of one or more of the structure and the apparatus in a predetermined way.

As used herein, the singular forms “a,” “an,” and “the” are intended to include the plural forms as well, unless the context clearly indicates otherwise. Additionally, as used herein, the terms “a,” “an,” “at least one,” and “one or more” are interchangeable, and in reference to a particular item, refer to a single instance of the particular item as well as multiple (e.g., more than one) instances of the particular item, unless context clearly indicates otherwise. For example, the terms “single” or “individual” following “a,” “an,” or “the” in reference to a particular item is a clear indication that the term “a,” “an,” or “the” refers to one and only one of the particular item.

As used herein, “and/or” includes any and all combinations of one or more of the associated listed items.

As used herein, the terms “vertical,” “longitudinal,” “horizontal,” and “lateral” are in reference to a major plane of a structure and are not necessarily defined by earth's gravitational field. A “horizontal” or “lateral” direction is a direction that is substantially parallel to the major plane of the structure, while a “vertical” or “longitudinal” direction is a direction that is substantially perpendicular to the major plane of the structure. The major plane of the structure is defined by a surface of the structure having a relatively large area compared to other surfaces of the structure.

As used herein, reference to a feature as being “over” an additional feature means and includes the feature being directly on top of, adjacent to (e.g., horizontally adjacent to, vertically adjacent to), underneath, or in direct contact with the additional feature. It also includes the element being indirectly on top of, adjacent to (e.g., horizontally adjacent to, vertically adjacent to), underneath, or near the additional feature, with one or more other features located therebetween. In contrast, when an element is referred to as being “on” or another element, there are no intervening features therebetween.

As used herein, the term “substantially” in reference to a given parameter, property, or condition means and includes to a degree that one of ordinary skill in the art would understand that the given parameter, property, or condition is met with a degree of variance, such as within acceptable tolerances. By way of example, depending on the particular parameter, property, or condition that is substantially met, the parameter, property, or condition may be at least 90.0 percent met, at least 95.0 percent met, at least 99.0 percent met, at least 99.9 percent met, or even 100.0 percent met.

As used herein, “about” or “approximately” in reference to a numerical value for a particular parameter is inclusive of the numerical value and a degree of variance from the numerical value that one of ordinary skill in the art would understand is within acceptable tolerances for the particular parameter. For example, “about” or “approximately” in reference to a numerical value may include additional numerical values within a range of from 90.0 percent to 110.0 percent of the numerical value, such as within a range of from 95.0 percent to 105.0 percent of the numerical value, within a range of from 97.5 percent to 102.5 percent of the numerical value, within a range of from 99.0 percent to 101.0 percent of the numerical value, within a range of from 99.5 percent to 100.5 percent of the numerical value, or within a range of from 99.9 percent to 100.1 percent of the numerical value.

Prostheses described herein may be used for augmenting tissue of the subject. For example, prosthetic breast implants are generally designed to be compatible with tissue behind the breast being augmented. Such compatibility is commonly maintained through use of a non-absorbable flexible envelope or shell. The flexible shell is typically made from silicone rubber, polyurethanes or polyolefins. These materials have the properties of being flexible and water impermeable. The shell generally comprises of a single layer of silicone rubber, polyethylene terephthalate (PET), or polytetrafluoroethylene (PTFE). Alternatively, the shell may consist of multiple layers of the materials identified above. A saline solution or a synthetic silicone gel is normally used as the filler for the flexible shell. Examples of such fillers are described in U.S. Pat. No. 5,344,451 (Dayton).

Prostheses described in accordance with the instant disclosure may also be used to facilitate tissue growth for reconstruction procedures. For example, one or more embodiments of the prosthesis may be used to facilitate breast reconstruction (e.g., following a mastectomy). The prosthesis may include a flexible shell that may be inserted within the subject (e.g., human). The prosthesis may be positioned in a submuscular placement (e.g., under the large pectoralis muscle) in which the flexible shell of the prosthesis may be filled with a saline solution. Additionally, the prosthesis may be positioned in a prepectoral placement (e.g., over the large muscle) in which the flexible shell may be initially filled with air. A prepectoral placement may involve a more sturdy (e.g., rigid) prosthesis due to the fragility of the skin overlying the prosthesis, so the prosthesis may include additional elements such as a mesh around the flexible shell. Accordingly, in some embodiments, the prosthesis and/or components thereof may be fluidly sealed (e.g., hermetically sealed, liquid-tight sealed).

To insert the prosthesis into a subject (e.g., mammal), a healthcare professional (e.g., surgeon) and/or machine (e.g., surgical robot) may flatten a prosthesis. The healthcare professional and/or machine may be provided a cutting tool (e.g., scalpel) for incising the subject. An incision may be formed within the subject such that the resulting incision has a height and width sized to receive the flattened prosthesis. The prosthesis may be inserted through the incision and behind the breast, mons pubis, buttocks, fascia, and/or pectoral muscle of the subject. The incision may then be closed (e.g., sutured, stapled, glued, and/or zipped). In one or more embodiments, a biocompatible fluid (e.g., saline solution) may be injected into the prosthesis after inserting the prosthesis into the subject and before closing the incision.

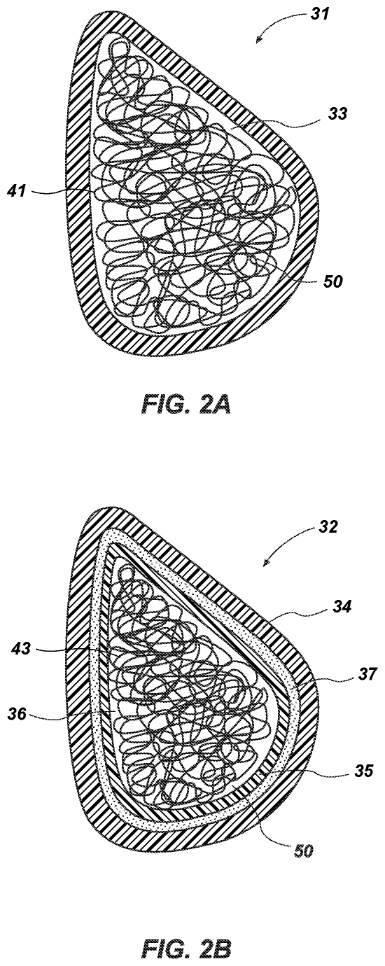

The instant disclosure incorporates a super elastic material (SEM) (e.g., super elastic alloy (SEA) and/or super elastic polymer (SEP)) into a prosthesis. For example, embodiments of the disclosure may utilize temperature-sensitive super elastic materials. In some embodiments, the prosthesis may include a super elastic material nest (e.g., FIG. 2A). In additional embodiments, the prosthesis may include a super elastic material web-like mesh (e.g., FIG. 2B). In additional embodiments, the prosthesis may include a super elastic material cage structure (e.g., FIG. 2C). The prosthesis may include one or more strands of super elastic material arranged (e.g., woven) to form the nest, the web-like mesh, and/or the cage structure. In some embodiments, the prosthesis may include a combination of a super elastic material cage structure, super elastic material web-like mesh, and/or super elastic material arranged in the shape of a nest.

In certain embodiments, prostheses described herein include a super elastic material disposed within and/or incorporated into a flexible shell. A saline solution or synthetic silicone gel may be used as fillers in conjunction with the super elastic material. Part of the interest in super elastic materials derives from their ability to “remember” shapes.

Prostheses in accordance with embodiments of the disclosure may provide certain benefits over conventional prostheses. For example, prostheses described herein may be collapsible for incision and expandable after placement within a subject, enabling a smaller incision for placement because the prostheses can be temporarily collapsed for insertion into the subject, and then return to the desired prostheses shape while inside of the subject. Embodiments of the prostheses described herein may also reduce the amount of scar tissue associated with the prostheses. In one or more embodiments, prostheses described herein may reduce or eliminate leakage from the prosthesis. Prostheses as described herein may offer an alternative to current silicone gel and saline implants that lead to concerns about diseases (e.g., autoimmune diseases) and cancer.

For super elastic alloys, the basis for this unique “shape memory” phenomena is due to the inherent phase transformation that occurs within the crystal structure of the alloy when it is cooled from its stronger, high temperature form (austenite) to its weaker, low temperature form (martensite). The inherent phase transformation leads to two unique properties which, in particular, are referred to as the “shape memory” effect and the “super-elasticity” effect. The metallurgical phenomena that leads to these effects is summarized, for example, in U.S. Pat. No. 4,505,767 (Quin), the contents of which are incorporated herein by this reference.

Described briefly, the shape memory effect occurs when a super elastic alloy element having an original shape is heated well above (e.g., at least about 10% above) the austenitic transition temperature and the super elastic alloy element is manipulated to a desired predetermined “remembered” shape. The super elastic alloy element may then be cooled below the austenitic transition temperature such that the super elastic alloy element transitions from its austenitic state to its martensitic state. Below the austenitic transition temperature, the super elastic alloy element may be deformed and will retain its deformed shape so long as the element remains in the martensitic state, i.e., so long as the element remains below the austenitic transition temperature. But when the super elastic alloy element is heated above the austenitic transition temperature and the super elastic alloy element returns to its austenitic state, the desired predetermined “remembered” shape is recovered. The super elasticity effect, on the other hand, occurs when a super elastic alloy element having an original shape or configuration is deformed at a temperature at about and/or slightly above (e.g., less than about 5% above) the austenitic transformation temperature. Deformation of the super elastic alloy element, while the element remains at a temperature at about or slightly above the austenitic transformation temperature, results in stress-induced formation of martensite. Because the martensite is formed while the element is in the austenitic temperature range, the martensite will automatically revert back to austenite once the deformation stress is removed. In reverting back to the austenitic phase, the desired predetermined “remembered” shape is recovered.

Alloys of nickel and titanium have been identified as possessing shape memory and super elasticity properties. The compositions and properties of such alloys and the manners of making them are well known in the metallurgical arts, as described in, for example, U.S. Pat. No. 3,174,851 (Buehler et al.), U.S. Pat. No. 3,351,463 (Rozner et al.), U.S. Pat. No. 3,403,238 (Beuhler et al.), U.S. Pat. No. 3,753,700 (Harrison et al.), and U.S. Pat. No. 3,832,243 (Donkersloot), the contents of each of which are incorporated herein by this reference. Other shape memory material compositions are disclosed in U.S. Pat. No. 9,062,141 (Jun. 23, 2015) to Goodrich et al., U.S. Pat. No. 9,789,231 (Oct. 17, 2017) to Goodrich, and U.S. Pat. No. 10,590,218 (Mar. 17, 2020) to Goodrich et al., the contents of each of which are incorporated herein by this reference in their entirety.

In particular, U.S. Pat. No. 4,283,233 (Goldstein et al.), the contents of which is incorporated herein by this reference, describes a method of modifying the transition temperature range—i.e., the temperature range over which the transition from martensite to austentite, and vice versa, occurs—of nickel-titanium based alloys so that the resulting super elastic alloys possess usefulness as prosthetic devices in humans. More specifically, Goldstein et al. describes the making of a super elastic alloy whose transition temperature range, or austenitic transition temperature, falls just below the subject's (e.g., mammal's) normal body temperature (T_{Body}), thus allowing for the shape memory effect and super elasticity effect to occur in conjunction with the prosthesis due to body heat. As a non-limiting example, the normal human body temperature (T_{Body}) of a human being is about 37 degrees Celsius (37° C.).

Super elastic polymers overall function similarly to super elastic alloys. However, with super elastic polymers, there is a hard phase with a high glass transition temperature (T_{g}) and a second, switching phase, with an intermediate or melting temperature (Tₘ) that enables the thermally responsive behavior. First, the super elastic polymers is heated above the highest thermal transition temperature (T_{Perm}) to establish the physical crosslinks responsible for the predetermined “remembered” (e.g., permanent) shape. Then, the super elastic polymer is cooled below the highest thermal transition temperature to set the physical crosslinks. Next, the temperature of the super elastic polymer is elevated higher than one of the high glass transition temperature (Tg) or the melting temperature (Tₘ), and a temporary shape can be induced by deforming the super elastic polymer. The temporary shape of the super elastic polymer can then be temporarily “remembered” by cooling the deformed state to a temperature below the utilized temperature (e.g., the high glass transition temperature (Tg)). When the deformed super elastic polymer is heated above the utilized temperature (e.g., the high glass transition temperature (Tg)), the super elastic polymer transforms back to its predetermined “remembered” (e.g., permanent) shape. To achieve shape memory properties, a polymer either has some degree of chemical crosslinking to form a “memorable” network or contains a finite fraction of hard regions serving as physical crosslinks.

In particular, U.S. Patent Pub. No. 2009/0248141 (Shandas et al.), incorporated herein by this reference, describes a method of tailoring the transition temperature of super elastic polymers to allow recovery at, above, or below the human body temperature of 37° C.

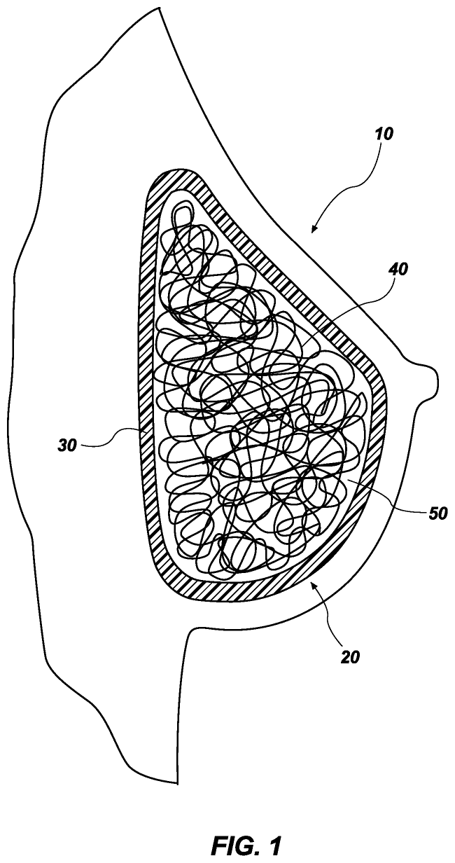

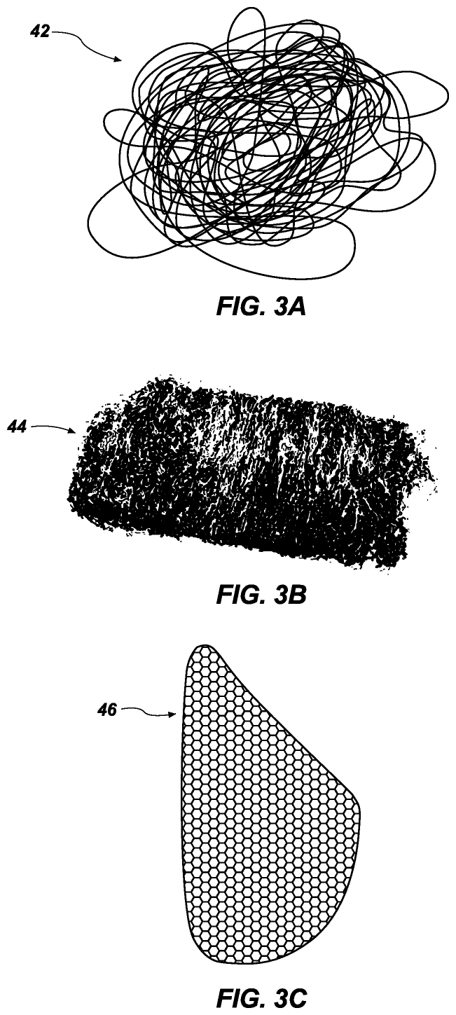

In FIG. 1, there is illustrated a cross-sectional view of a human breast 10 with an embodiment of the prosthesis 20 surgically implanted therein. As shown in FIG. 1, the prosthesis 20 includes a flexible shell 30, a support structure 40 within the flexible shell 30, and/or a filler material 50 within the flexible shell 30. While the prosthesis 20 is illustrated in FIG. 1 as including a flexible shell 30, a support structure 40, and a filler material 50, one or more of the flexible shell 30, the support structure 40, and the filler material 50 may be omitted from the prosthesis 20. In one or more embodiments, the prosthesis 20 includes only the flexible shell 30. In additional embodiments, the prosthesis 20 includes only the support structure 40. Additionally, the filler material 50 is optional. Thus, in some embodiments, the prosthesis 20 includes a flexible shell 30. In additional embodiments, the prosthesis includes a support structure 40. In further embodiments, the prosthesis 20 includes a flexible shell 30 and a support structure 40 within the flexible shell 30. In one or more embodiments, the prosthesis 20 may be filled with a filler material 50. Additionally, the prosthesis 20 illustrated in FIG. 1 and additional embodiments of prostheses described herein may include one or more of the flexible shells 31, 32 described below with reference to FIGS. 2A and 2B. Furthermore, the prosthesis 20 illustrated in FIG. 1 and additional embodiments of prostheses described herein may include one or more of the support structures 42, 44, 46 described below with reference to FIGS. 3A-3C.

FIGS. 2A and 2B illustrate embodiments of flexible shells 31 and 32 (e.g., the flexible shell 30 of FIG. 1). Referring collectively to FIGS. 2A and 2B, the flexible shell 31, 32 is sized and shaped for augmenting, replacing, and/or reconstructing tissue (e.g., breast tissue, buttocks tissue, or mons pubis tissue). In one or more embodiments, the flexible shell 31, 32 includes a support structure (e.g., the support structure 40 of FIG. 1) within the flexible shell 31, 32. The flexible shell 31, 32 may form a fluid-tight (e.g., air-tight, liquid tight) seal. For example, the flexible shell 31, 32 may form a fluid-tight (e.g., hermetic) seal to contain the filler material 50 within the flexible shell 31, 32 and to prevent the filler material 50 from leaking into the subject's surrounding tissue.

The flexible shell 31, 32 may be made of and/or include a biocompatible elastomer (e.g., silicone rubber, polyether, polyester urethane, polyether polyester copolymer, polypropylene oxide, polyethylene terephthalate (PET), and/or polytetrafluoroethylene (PTFE)). The flexible shell 31, 32 being made of a biocompatible elastomer to facilitate expansion and contraction of the flexible shell 31, 32 to accommodate changes in shape of the prosthesis due to the super elastic material. The flexible shell 31, 32 may also be made of and/or include a super elastic material. For example, the flexible shell 31, 32 may include a super elastic alloy (e.g., nitinol), and/or a super elastic polymer (e.g., polyether, polyacrylate, polyamide, polysiloxane, polyurethane, polyethylene, methyl-methacrylate (MMA), polyethylene glycol (PEG), polyethylene glycol dimethacrylate (PEGDMA), polyether amide, polyether ester, or urethane-butadiene copolymer).

Referring now to FIG. 2A, in some embodiments, the flexible shell 31 defines an internal cavity 33 within the flexible shell 31. In some embodiments, the flexible shell 31 also includes a support structure 41 (e.g., the support structure 40 of FIG. 1, and/or support structures 42, 44, 46 of FIGS. 3A-3C) within the internal cavity 33. For example, the support structure may include any of the support structures described below with reference to FIGS. 3A-3C disposed within the internal cavity 33. The exterior surface of the flexible shell 31 may have a rough texture (e.g., areas or features that are elevated or recessed relative to other areas or features). As non-limiting examples, a rough texture may include surface corrugations, islands that are elevated relative to surrounding areas. As another non-limiting example, a rough texture may include a surface including different particle sizes similar to sandpaper. A rough texture may facilitate growth of the subject's tissue.

Referring now to FIG. 2B, embodiments of the flexible shell 32 may include an outer shell 34, an inner shell 35 defining an internal cavity 36, and a web-like mesh 37 of super-elastic material interposed between the outer shell 34 and the inner shell 35. For example, the web-like mesh 37 may be in physical contact with and/or secured to an inner surface of the outer shell 34 and/or an exterior surface of the inner shell 35. In some embodiments, the flexible shell 32 also includes a support structure 43 (e.g., the support structure 40 of FIG. 1, and/or support structures 42, 44, 46 of FIGS. 3A-3C) within the internal cavity 36. For example, the support structure may include any of the support structures described below with reference to FIGS. 3A-3C disposed within the internal cavity 36. The flexible shell 32 and/or an exterior surface of the flexible shell 32 (e.g., the exterior surface of the outer shell 34) may have a rough texture (e.g., areas or features that are elevated or recessed relative to other areas or features). As non-limiting examples, a rough texture may include surface corrugations, islands that are elevated relative to surrounding areas. As another non-limiting example, a rough texture may include a surface including different particle sizes similar to sandpaper. A rough texture may facilitate growth of the subject's tissue.

In certain embodiments, the flexible shell 32 may also include the filler material 50 contained inside of a cavity within the flexible shell 32 (e.g., within the web-like mesh 37 between the outer shell 34 and inner shell 35 and/or within the internal cavity 36 of the inner shell 35). The filler material 50 may include a super elastic material and/or a biocompatible solution, such as saline. In some embodiments, the filler material comprises a super elastic material. In additional embodiments, the filler material comprises a saline solution. In further embodiments, the filler material comprises a combination of super elastic material and saline solution.

FIGS. 3A-3C illustrate embodiments of support structures 42, 44, 46 (e.g., the support structures 40 of FIG. 1). Referring collectively to FIGS. 3A-3C, the support structure 42, 44, 46 may be made of and/or include at least one strand of a super elastic material (e.g., a temperature-sensitive super elastic material). For example, the support structure 42, 44, 46 may include a super elastic alloy (e.g., nitinol), and/or a super elastic polymer (e.g., polyether, polyacrylate, polyamide, polysiloxane, polyurethane, polyethylene, methyl-methacrylate (MMA), polyethylene glycol (PEG), polyethylene glycol dimethacrylate (PEGDMA), polyether amide, polyether ester, or urethane-butadiene copolymer). The support structure 42, 44, 46 may be sized and shaped for augmenting, replacing, or reconstructing tissue (e.g., breast tissue, buttocks tissue, or mons pubis tissue). In some embodiments, the support structure 42, 44, 46 may include one or more strands of super elastic material. For example, in some embodiments, the support structure 42, 44, 46 may include a single strand of super elastic material. In additional embodiments, the support structure 42, 44, 46 may include multiple strands of super elastic material.

The transition temperature (“Tₜ”) of the support structure 42, 44, 46 may be tailored to be slightly below the subject's (e.g., human's) body temperature. For example, the transition temperature of the support structure 42, 44, 46 may be tailored to be about 37° C. The desired transition temperature (Tt) may facilitate activation of both the shape memory effect and the super elasticity properties of the support structure 42, 44, 46. For example, in embodiments in which the support structure 42, 44, 46 comprises a super elastic alloy material, the transition temperature (Tₜ) may be the austenitic transition temperature. In embodiments in which the super elastic alloy support structure 42, 44, 46 comprises a super elastic polymer material, the transition temperature (Tₜ) may be either the high glass transition temperature (T_{g}) or the intermediate (e.g., melting) temperature (Tₘ).

The support structure 42, 44, 46 may be formed into a desired predetermined “remembered” shape at a temperature above the transition temperature (Tₜ), and then the support structure 42, 44, 46 may be cooled below the transition temperature (Tₜ). The support structure 42, 44, 46 may then be deformed, if desired, for insertion into the flexible shell 30 (FIG. 1). The support structure 42, 44, 46 may then be further deformed, if desired, for the actual implanting of the prosthesis 20 (FIG. 1) under the skin. Once the prosthesis 20 (FIG. 1) has been implanted, the body temperature of the subject may warm the support structure 42, 44, 46, and/or the flexible shell 30 (FIG. 1) to a temperature above the transition temperature (Tₜ), which may result in the support structure 42, 44, 46 (and, optionally, the flexible shell 30 (FIG. 1)) returning to the desired predetermined “remembered” shape.

The support structure 42, 44, 46 may comprise one or more strands of super elastic material exhibiting any desired and/or suitable dimensions for the prosthesis 20 of FIG. 1. As non-limiting examples, the super elastic material wire may be about 4 American Wire Gauge (AWG) and smaller. For example, the super elastic material wire may be from about 4 AWG (5.189 mm diameter) to about 30 AWG (0.255 mm diameter), such as from about 6 AWG (4.1148 mm diameter) to about 20 AWG (0.812 mm diameter), from about 9 AWG (2.90576 mm diameter) to about 15 AWG (1.45034 mm diameter), and more particularly from about 10 AWG (2.58826 mm diameter) to about 13 AWG (1.8288 mm diameter), such as about 10 AWG (2.58826 mm diameter). In some embodiments, the prosthesis comprises multiple strands of super elastic material. In some embodiments, a first strand of super elastic material of the multiple strands of super elastic material may exhibit a first diameter, and a second strand of super elastic material of the multiple strands of super elastic material may exhibit a second diameter. In one or more embodiments, the first diameter is the same as the second diameter. In additional embodiments, the first diameter is different than the second diameter.

FIG. 3A illustrates a support structure 42 (e.g., the support structure 40 of FIG. 1), in accordance with embodiments of the disclosure. The support structure 42 includes at least one strand of super elastic material configured (e.g., arranged) in a nest shape. The nest shape may somewhat resemble the shape of a bird's nest.

FIG. 3B illustrates another support structure 44 (e.g., the support structure 40 of FIG. 1), in accordance with embodiments of the disclosure. The support structure 44 may include multiple intertwined (e.g., interwoven) strands of superelastic material arranged in the shape of a web-like mesh. In one or more embodiments, the web-like mesh may somewhat resemble the shape and configuration of a typical pad of steel wool. In additional embodiments, the web-like mesh may somewhat resemble the shape of a spider's web.

FIG. 3C illustrates an additional support structure 46 (e.g., the support structure 40 of FIG. 1), in accordance with embodiments of the disclosure. The support structure 46 comprising the at least one super elastic strand configured (e.g., arranged) to form a planar pattern (e.g., hexagonal, triangular, rectangular, square, trapezoidal) that is then manipulated to form a cage structure in a desired shape. By way of non-limiting example, the cage structure may exhibit an ellipsoid shape (e.g., an egg-shape), a spherical shape, a conical shape, a frusto-conical shape, a pyramid shape, a frusto-pyramidal shape, a breast shape, a shape representing a “divot” in a body wound or cavity, or a combination of two or more of the foregoing shapes. In some embodiments, the support structure 46 may include a combination of the nest, web-like mesh, and/or the cage structure. For example, the support structure 46 may comprise at least one super elastic material strand entwined and/or woven to form a cage structure and at least one strand of super elastic material arranged in a nest shape within the cage structure.

When the support structure 42, 44, 46 is placed in a subject without a shell to shape the subject's tissue (e.g., breast), the support structure 42, 44, 46 may fill with the subject's bodily fluids, and no filler material 50 (FIG. 1), such as an external source of fluid need be administered. Additionally or alternatively, the fluid (e.g., sterile normal saline) may be first administered and then an exchange of fluids with the body then takes place.

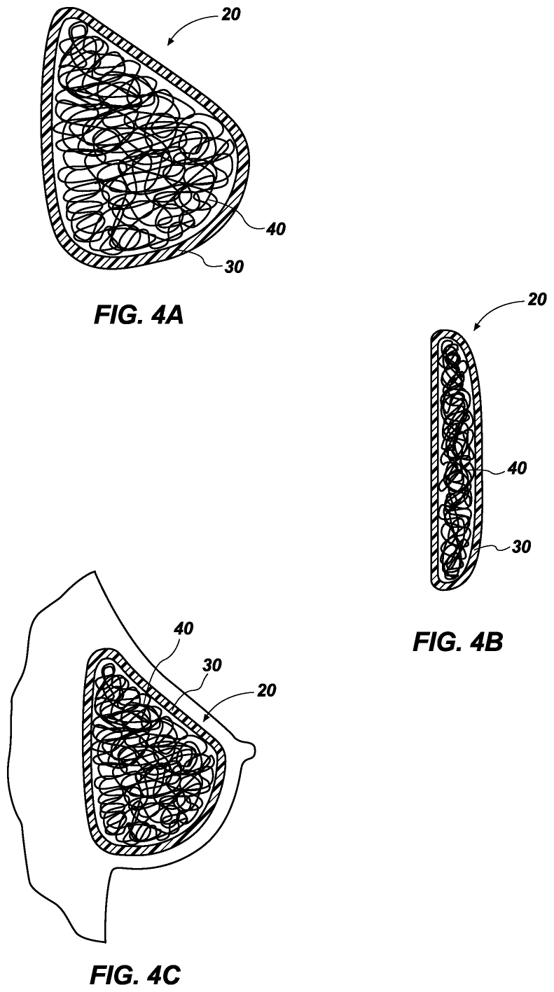

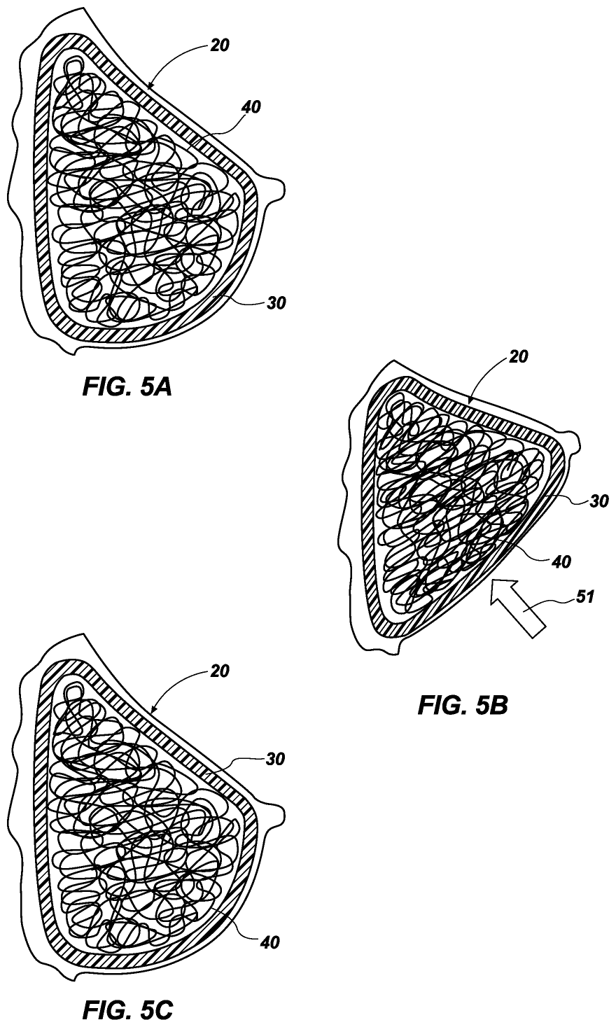

FIGS. 4A-C and 5A-C illustrate a sequence showing the shape memory effect properties of prostheses, in accordance with embodiments herein. For simplicity, FIGS. 4A-4C and 5A-5C refer to the prosthesis 20 of FIG. 1, although the shape memory effect properties may apply to any prosthesis that includes shape memory materials. For example, FIGS. 4A-C and FIGS. 5A-C may refer to a prosthesis that includes a flexible shell (e.g., the flexible shell 31 (FIG. 2A), the flexible shell 32 (FIG. 2B)), and/or a support structure (e.g., the support structure 42 (FIG. 3A), the support structure 44 (FIG. 3B), and/or the support structure 46 (FIG. 3C)), and the prosthesis may also include a filler material (e.g., the filler material 50 (FIG. 1)).

FIGS. 4A-C illustrate a sequence showing the shape memory effect properties of the prosthesis 20. In some embodiments, the flexible shell may comprise a material that exhibits a shape memory effect. In additional embodiments, the support structure 40 may comprise a material that exhibits a shape memory effect. In further embodiments, each of the flexible shell 30 and the support structure 40 may comprise a material that exhibit shape memory effects.

FIG. 4A illustrates an embodiment of the prosthesis 20 in the desired predetermined “remembered” shape (e.g., the natural shape of the breast tissue) external to the subject (e.g., human). The prosthesis 20 may comprise one or more super elastic materials exhibiting shape memory effect properties.

Initially, the prosthesis 20, including the flexible shell 30 and/or the support structure 40, may be heated above (e.g., about 10% or more above) the transition temperature (Tₜ) of the super elastic material. As previously discussed, the transition temperature (Tₜ) may be tailored to be the subject's (e.g., human's) normal internal body temperature (e.g., about 37° C.).

In embodiments in which the super elastic material includes super elastic alloys, the super elastic alloy of the prosthesis 20 is heated to a temperature well above (e.g., about 10% or more above) the austenitic transition temperature and the prosthesis 20 is oriented in a desired predetermined “remembered” shape. In response to the prosthesis 20 being in the desired predetermined “remembered” shape, the prosthesis 20 is cooled below the austenitic transition temperature of the super elastic alloy. Accordingly, the predetermined “remembered” shape may be “locked-in” to be the default shape of the prosthesis 20 when the super elastic material is about or above the austenitic transition temperature. The prosthesis 20 can then be temporarily deformed. When the deformed prosthesis 20 is heated above the austenitic transition temperature, the prosthesis 20 transforms back to its predetermined “remembered” shape.

In embodiments in which the super elastic material includes super elastic polymers, the super elastic polymer of the prosthesis 20 is heated above the highest thermal transition temperature (T_{Perm}) to establish the physical crosslinks responsible for the predetermined “remembered” (e.g., permanent) shape. Then, the prosthesis 20 is cooled below the highest thermal transition temperature of the super elastic polymer to set the physical crosslinks. Next, the prosthesis is heated above (e.g., about 10% or more above) either the super elastic polymer's high glass transition temperature (T_{g}) or the melting temperature (Tₘ), and a temporary shape can be induced by deforming the prosthesis 20. The temporary shape of the super elastic polymer can then be temporarily “remembered” by cooling the prosthesis 20 in the deformed state to a temperature below the utilized transition temperature (e.g., the melting temperature (Tₘ)). When the deformed prosthesis 20 is heated above the utilized temperature (e.g., the melting temperature (Tₘ)) of the super elastic polymer, the prosthesis 20 transforms back to its predetermined “remembered” (e.g., permanent) shape.

In embodiments in which the super elastic material includes both super elastic polymers and super elastic alloys, the super elastic material of the prosthesis 20 may be heated about 10% or more above the transition temperature (Tₜ) of the composite material. At temperatures well above (e.g., about 10% or more above) or more above the transition temperature, the flexible shell 30 and/or the support structure 40 may be formed into the desired predetermined “remembered” (e.g., undeformed) shape. In response to the prosthesis 20 (e.g., the flexible shell 30 and/or the support structure 40) being in the desired predetermined “remembered” shape, the temperature of the flexible shell 30 and/or the support structure 40 may be lowered below the transition temperature (Tₜ). Accordingly, the predetermined “remembered” shape may be “locked-in” to be the default shape when the super elastic material is about or above the transition temperature. The prosthesis 20, including the flexible shell 30 and/or the support structure 40, may remain in the desired predetermined “remembered” shape at temperatures below the transition temperature. Below the transition temperature, the super elastic polymer material may include some degree of chemical crosslinking or a finite fraction of hard regions serving as physical crosslinks. Below the transition temperature, the super elastic alloy material is in the martensitic phase.

As illustrated in FIG. 4B, the prosthesis 20 may, if desired, be flattened or otherwise deformed prior to implantation to facilitate the implantation process. For example, the temperature of the prosthesis 20 (e.g., the flexible shell 30 and/or the support structure 40) may remain below the transition temperature (Tₜ) and/or the subject's internal body temperature (T_{Body}). In embodiments in which the prosthesis 20 includes a super elastic alloy material, the prosthesis 20 may be deformed while the super elastic alloy material is in the martensite phase. For example, a support structure 40 including super elastic alloy material may be deformed at a temperature below the austenitic transformation temperature. In embodiments in which the prosthesis 20 includes a super elastic polymer material, the prosthesis 20 may be deformed into the temporary shape at a temperature at about or above one of the high glass transition temperature (T_{g}) or the melting temperature (Tₘ). Then, the prosthesis 20 including super elastic polymer material may be cooled to a temperature below the utilized transition temperature (e.g., the high glass transition temperature (T_{g}) or the melting temperature (Tₘ)), and the super elastic polymer material may retain its deformed shape.

As illustrated in FIG. 4C, the prosthesis 20 (e.g., the flexible shell 30 and/or the support structure 40) may be implanted behind the breast, fascia, pectoral muscle tissue. At this point, the heat from the human body may raise the temperature of the prosthesis 20 above the transition temperature (Tₜ), which may be at or about the subject's (e.g., human's) normal internal body temperature (e.g., at or about 37° C.). In response to the super elastic material of the prosthesis being at about or above the transition temperature (Tₜ), the prosthesis 20 may return to the predetermined “remembered” (e.g., permanent, un-deformed) shape illustrated in FIG. 3A. For example, the support structure 40 and/or the flexible shell 30 may include super elastic material that may be above the transition temperature (Tₜ). In embodiments in which the prosthesis 20 comprises super elastic alloy material, the super elastic alloy material may return to the austenitic phase, and the predetermined “remembered” (e.g., permanent) shape is recovered. In embodiments in which the prosthesis 20 comprises super elastic polymer material, the super elastic polymer material may be above the utilized transition temperature (e.g., the high glass transition temperature (T_{g}) or the melting temperature (Tₘ)), causing the prosthesis 20 to return to the predetermined “remembered” (e.g., permanent) shape. Accordingly, the shape memory effect of the prosthesis 20 may be triggered, resulting in the prosthesis 20 recovering the predetermined “remembered” shape previously illustrated in FIG. 4A.

FIGS. 5A-5C illustrate a sequence of acts showing the super elasticity properties of the prosthesis 20 that has been implanted within a subject (e.g., a human). The prosthesis 20 illustrated in FIGS. 5A-5C includes a super elastic material, the properties of which are similar to those described above. Additionally, because the prosthesis 20 illustrated in FIGS. 5A-5C has been implanted within a subject, the prosthesis 20 may be at a temperature about or above (e.g., slightly above) the transition temperature (Tₜ).

Specifically, FIG. 5A illustrates a fully implanted prosthesis 20 similar to FIG. 4C. The prosthesis 20 (e.g. flexible shell 30 and/or support structure 40) may be at about or above the subject's normal internal body temperature (e.g., about 37° C.), which may be at or above the transition temperature (Tₜ). For example, in embodiments in which the prosthesis 20 comprises a super elastic alloy material, the super elastic alloy material is in the austenitic phase. In embodiments in which the prosthesis 20 comprises a super elastic polymer material, the super elastic polymer material is in the predetermined “remembered” (e.g., permanent) shape and the temperature of the support structure 40 is at about or slightly above a utilized transition temperature (e.g., the high glass transition temperature (T_{g}) or the melting temperature (Tₘ)).

In FIG. 5B, the prosthesis 20 is illustrated as being manipulated slightly by a force 51 in the upward direction. Deformation of the prosthesis 20 (e.g., the flexible shell 30 and/or the support structure 40) comprising super elastic material under the cited conditions results in stress within the super elastic material. For example, in embodiments in which the prosthesis 20 comprises super elastic alloy material, the stresses within the super elastic alloy material may result in in a temporary phase transformation (e.g., stress-induced martensite). In embodiments in which the prosthesis 20 comprises a super elastic polymer material, the stresses within the super elastic polymer material may result temporary deformation to the prosthesis 20. Because the temperature of the super elastic material of the prosthesis 20 remains within the subject at or above the transition temperature (Tₜ), but not far enough above the transition temperature (Tₜ) to re-program the super elastic material, the prosthesis 20 will return to the predetermined “remembered” (e.g., permanent) shape, as illustrated in FIG. 5C.

In FIG. 5C, the prosthesis 20 is illustrated as having returned to the predetermined “remembered” shape. For example, in embodiments in which the prosthesis 20 comprises super elastic alloy material, the stress-induced martensite will transform back to the austenitic phase. Similarly for super elastic polymers, because the temperature of the prosthesis 20 remains above the utilized transition temperature (e.g., the high glass transition temperature (T_{g}) or the melting temperature (Tₘ)), the deformed polymer shape returns to the predetermined “remembered” (e.g., permanent) shape. Accordingly, the prosthesis 20 and/or the subject's tissue may recover the shape previously illustrated in FIG. 5A (or FIG. 4C) once the deforming load is removed.

The foregoing description illustrates the basic properties of the prosthesis of the instant disclosure. Various other modes for carrying out the invention, such as variations in the composition of the super elastic material mesh, are contemplated as being within the scope of the following claims particularly pointing out and distinctly claiming the subject matter which is regarded as the invention.

Once being apprised of this disclosure, one of ordinary skill in the art will be able to make and use the devices described herein.

What is claimed is:

1. A prosthesis for implantation beneath skin of a subject, the prosthesis comprising a support structure sized and shaped for augmenting, replacing, or reconstructing tissue of the subject, the support structure comprising at least one strand of super elastic material.

2. The prosthesis of claim 1, wherein the support structure is arranged in a nest shape.

3. The prosthesis of claim 1, wherein the support structure comprises a cage structure.

4. The prosthesis of claim 1, wherein the support structure comprises:
a cage structure; and
the at least one strand of super elastic material arranged in a nest shape within the cage structure.

5. The prosthesis of claim 1, further comprising:
a flexible shell defining an internal cavity within the flexible shell, the flexible shell being sized and shaped for augmenting, replacing, or reconstructing breast tissue of the subject; wherein the at least one strand of super elastic material is arranged in a nest shape and disposed within the internal cavity of the flexible shell.

6. The prosthesis of claim 5, wherein the flexible shell comprises silicone rubber.

7. The prosthesis of claim 5, wherein the flexible shell comprises an exterior surface having a rough texture.

8. The prosthesis of claim 1, wherein the super elastic material comprises nitinol.

9. The prosthesis of claim 1, wherein the super elastic material comprises a super elastic polymer.

10. The prosthesis of claim 9, wherein the super elastic polymer comprises polyethylene.

11. A prosthesis for implantation under skin and/or beneath a breast, fascia, or pectoral muscle of a subject, the prosthesis comprising:
a support structure sized and shaped for augmenting, replacing, or reconstructing tissue of a subject, the support structure comprising a super elastic material configured to selectively transition to a predetermined shape.

12. The prosthesis of claim 11, wherein the support structure comprises web-like mesh of super elastic material having a surface contour sized and shaped for augmenting, replacing, or reconstructing tissue of the subject.

13. The prosthesis of claim 11, wherein the support structure comprises at least one strand of super elastic material.

14. The prosthesis of claim 13, wherein the at least one strand of super elastic material exhibits a diameter of from about 6 American Wire Gauge (AWG) to about 20 AWG.

15. The prosthesis of claim 11, wherein the support structure comprises a temperature-sensitive super elastic material configured to transition to the predetermined shape in response to being at about a transition temperature of the super elastic material.

16. The prosthesis of claim 11, wherein the support structure comprises multiple strands of super elastic material.

17. The prosthesis of claim 16, wherein the multiple strands of super elastic material comprises:
a first strand of super elastic material exhibiting a first diameter; and
a second strand of super elastic material exhibiting a second diameter, different from the first diameter.

18. A prosthesis for implantation beneath a breast, fascia, or pectoral muscle of a subject, the prosthesis comprising:
an elastomeric outer shell, the outer shell being sized and shaped for augmenting, replacing, or reconstructing breast tissue of the subject;
an elastomeric inner shell positioned within the elastomeric outer shell, the elastomeric inner shell defining an interior cavity; and
a web-like mesh of super elastic material interposed between the elastomeric outer shell and the elastomeric inner shell.

19. The prosthesis of claim 18, further comprising a support structure within the elastomeric inner shell, the support structure comprising a super elastic material.

20. A method of implanting a breast prosthesis comprising a super elastic material into a subject, the method comprising:
flattening a prosthesis, the prosthesis comprising:
an elastomeric outer shell defining a cavity therein, the elastomeric outer shell being sized and shaped for augmenting or replacing breast tissue of the subject, and
a support structure within the elastomeric outer shell, the support structure comprising a super elastic material configured to transition to a predetermined shape in response to the super elastic material being at about 37 degrees Celsius;
incising the subject such that the resulting incision has a height and width sized to receive the flattened prosthesis;
inserting the prosthesis behind a breast, fascia, or pectoral muscle of the subject through the incision; and
closing the incision.
